(19) United States
(12) Patent Application Publication
(10) Pub. No.: US 2004/0022762 A1
(43) Pub. Date: Feb. 5, 2004

(54) Use of il-8 protein modulators in the treatment of viral infections

(76) Inventors: Susan B Dillon, Wayne, PA (US); Ruth Tal-Singer, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/381,066

(22) PCT Filed: Sep. 25, 2001
(86) PCT No.: PCT/US01/30222

(30) Foreign Application Priority Data
Sep. 25, 2000 (US) 60234914

(51) Int. Cl. A61K 45/00; A61K 39/395; A61K 38/20

(52) U.S. Cl. 424/85.2; 424/145.1

(57) ABSTRACT

The present invention is directed to the novel use of an IL-8 protein modulator for the treatment of human virus infections and associated symptom exacerbations.

### FIELD OF THE INVENTION

This invention relates to the use of IL-8, GROα, GROβ, GROγ, NAP-2, and ENA-78 protein modulators in the treatment of viral infections.

### BACKGROUND OF THE INVENTION

Human rhinovirus (HRV), the most frequent cause of the common cold, is increasingly associated with more serious sequelae including exacerbations of asthma, chronic bronchitis, COPD, otitis media, and sinusitis. Recent published studies in adults and adolescents, using PCR to assist in viral detection, have shown that up to 50 to 80% of asthma exacerbations are associated with upper respiratory tract virus infection, and that rhinovirus is the most common cold virus.

HRV infects nasal epithelial cells. Recent evidence suggests the virus may also infect bronchial epithelium. Prodromal cold symptoms are apparent within 24 hours post-infection, peak on days 2 through 5, and resolve within 7 to 14 days. However, the effects can be more protracted in some individuals. While the virus may clear, symptoms often persist. Symptoms are believed to arise more from the host's response to infection than an acute cytotoxic effect, since only a small fraction of upper respiratory epithelial cells are demonstrably infected, and there is minimal epithelial cell damage. Increased intranasal levels of kinins, IL-1, IL-8, IL-6, IL-11, and neutrophils are found in normal individuals infected with rhinoviruses. A correlation between IL-8 concentration in nasal secretions with local myeloperoxidase level as well as symptom severity has been demonstrated in several recent studies. Intranasal concentrations of IL-1 and IL-6 have been correlated with symptom severity as well. Experimental rhinovirus infection also results in enhanced immediate and late phase allergic reactions, and in increased infiltration of T lymphocytes and eosinophils into the lower airways. In atopics and asthmatics, these effects persist for up to 2 months post-infection. Human bronchial epithelial cell lines have been shown to produce IL-1, IL-6, IL-8, IL-11 and GM-CSF in response to rhinovirus infection. Early production of cytokines by rhinovirus-infected epithelial cells may therefore be responsible for triggering recruitment of neutrophils, T-cells and activated eosinophils into the upper and lower airways.

Additionally, IL-1, IL-6 and IL-8 are also produced in response to infection with other respiratory viruses (influenza, respiratory syncytial virus) which can cause the common cold and associated sequelae.

Many different names have been applied to Interleukin-8 (IL-8), such as neutrophil attractant/activation protein-1 (NAP-1), monocyte derived neutrophil chemotactic factor (MDNCF), neutrophil activating factor (NAF), and T-cell lymphocyte chemotactic factor. Interleukin-8 is a chemoattractant for neutrophils, basophils, and a subset of T-cells. It is produced by a majority of nucleated cells including macrophages, fibroblasts, endothelial and epithelial cells exposed to TNF, IL-1α, IL-1β or LPS, and by neutrophils themselves when exposed to LPS or chemotactic factors such as FMLP. M. Baggiolini et al., J. Clin. Invest. 84, 1045 (1989); J. Schroder et al, J. Immunol. 139, 3474 (1987) and J. Immunol. 144, 2223 (1990); Strieter et al., Science 243, 1467 (1989) and J. Biol. Chem. 264, 10621 (1989); Cassatella et al., J. Immunol. 148, 3216 (1992).

GROα, GROβ, GROγ and NAP-2 also belong to the chemokine family. Like IL-8, these chemokines have also been referred to by different names. For instance GROα, β, γ have been referred to as MGSAα, β and γ respectively (Melanoma Growth Stimulating Activity), see Richmond et al., J. Cell Physiology 129, 375 (1986) and Chang et al., J. Immunol. 148, 451 (1992). All of the chemokines of the α-family which possess the ELR motif directly preceding the CXC motif bind to the IL-8 B receptor (CXCR2).

IL-8, GROα, GROβ, GROγ, NAP-2, and ENA-78 stimulate a number of functions in vitro. They have all been shown to have chemoattractant properties for neutrophils, while IL-8 and GROα have demonstrated T-lymphocytes, and basophilic chemotactic activity. In addition IL-8 can induce histamine release from basophils from both normal and atopic individuals. GRO-α and IL-8 can, in addition, induce lysozomal enzyme release and respiratory burst from neutrophils. IL-8 has also been shown to increase the surface expression of Mac-1 (CD11b/CD18) on neutrophils without de novo protein synthesis.

In vitro, IL-8, GROα, GROβ, GROγ and NAP-2 induce neutrophil shape change, chemotaxis, granule release, and respiratory burst, by binding to and activating receptors of the seven-transmembrane, G-protein-linked family, in particular by binding to IL-8 receptors, most notably the IL-8β receptor (CXCR2). Thomas et al., J. Biol. Chem. 266, 14839 (1991); and Holmes et al., Science 253, 1278 (1991).

Two high affinity human IL-8 receptors (77% homology) have been characterized: IL-8Rα, which binds only IL-8 with high affinity, and IL-8Rβ, which has high affinity for IL-8 as well as for GROα, GROβ, GROγ and NAP-2. See Holmes et al., supra; Murphy et al., Science 253, 1280 (1991); Lee et al., J. Biol. Chem. 267, 16283 (1992); LaRosa et al., J. Biol. Chem. 267, 25402 (1992); and Gayle et al., J. Biol. Chem. 268, 7283 (1993).

Interference with the biochemical processes of epithelial cells resulting from virus infection represents a viable new therapeutic target for an IL-8 modulator. The present invention is directed to the novel discovery of treatment of chemokine-mediated diseases through this therapeutic target.

### SUMMARY OF THE INVENTION

The present invention provides for a method of treating a chemokine mediated disease, wherein the chemokine is one which binds to an IL-8α or β receptor and which method comprises administering an effective amount of an IL-8 protein modulator selected from the group consisting of an IL-8 antibody, an IL-8 receptor antagonist antibody, an IL-8 receptor peptide and a modified IL-8 peptide. The present invention further relates to the use of such IL-8 protein modulators for the treatment, including prophylaxis and prevention/reduction of the severity of the underlying condition, of a virus infection including but not limited to human rhinovirus, enteroviruses, coronavirus, influenza virus, parainfluenza virus, respiratory syncytial virus, and adenovirus, in a human in need thereof, which method comprises administering to said human an effective amount of such IL-8 protein modulators.

### DETAILED DESCRIPTION OF THE INVENTION

All publications, including but not limited to patents and patent applications, cited in this specification are herein incorporated by reference as though fully set forth.

IL-8 and other cytokines affect a wide variety of cells and tissues and these cytokines as well as other leukocyte-derived cytokines are important and critical inflammatory mediators responsible for the symptoms of many viral infections. The inhibition of these cytokines is of benefit in controlling, reducing and alleviating many of these symptoms of respiratory viral infection. In addition, the present invention is directed to the treatment of symptoms caused by viral infection in a human which is caused by the human rhinovirus, other enterovirus, coronavirus, herpesviruses, influenza virus, parainfluenza virus, respiratory syncytial virus or an adenovirus. In addition, the present invention is directed to respiratory viral infections which exacerbate underlying chronic conditions such as asthma, chronic bronchitis, chronic obstructive pulmonary disease, otitis media, and sinusitis. It should also be noted that the respiratory viral infection treated herein may be associated with a secondary bacterial infection, such as otitis media, sinusitis or pneumonia.

The present invention will demonstrate that IL-8 modulators having antagonist activity, where the modulator is a protein, i.e., IL-8 protein modulators, are useful in the treatment of symptoms associated with respiratory viral infection and prevention/reduction of the severity of exacerbations of underlying conditions, including asthma and otitis media, COPD, sinusitis, chronic bronchitis, etc., among others.

As used herein, the term “IL-8 protein modulators” includes antibodies and peptides which have IL-8 antagonist or IL-8 receptor antagonist activity.

Suitable IL-8 protein modulators are well known in the art, and an assay for determining IL-8 inhibition is also readily available. Anti-IL-8 antibodies are disclosed in U.S. Pat. Nos. 6,025,158; 5,702,946; 5,677,426; PCT Publication Nos. WO99/37779; WO98/37200; WO98/24893; WO98/24884; WO97/01354; WO96/33735; WO96/34096; WO96/02576; WO95/23865; WO93/02108; WO92104372; WO92/06697; and European Pat. No. EP 761688. Anti-IL-8 receptor antibodies are disclosed in U.S. Pat. Nos. 5,543,503; 5,440,021; and PCT Publication Nos. WO95/25126 and WO95/07934. IL-8 receptor peptides are disclosed in Japanese Pat. No. JP 06100595. Modified IL-8 peptides are disclosed in WO91/08231.

The term “anti-IL8 antibody” as used herein is defined as any antibody capable of binding to IL8. The term “anti-IL8 receptor antibody” as used herein is defined as any antibody capable of binding to either the IL-8Rα receptor or the IL-8Rβ receptor that by binding, inhibits receptor activity. The definition includes antibodies of all immunoglobulin types, such as IgG, IgA, IgM, IgD and IgE, and fragments thereof, and includes antibodies and antibody fragments of all origins, such as polyclonal antibodies, monoclonal antibodies, humanized antibodies and human antibodies produced in transgenic animals or transgenic animal cell culture.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′).sub.2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1)single-chain Fv (scFv) molecules (2)single chain polypeptides containing only one light chain variable domain, or a fragment thereof that contains the three CDRs of the light chain variable domain, without an associated heavy chain moiety and (3)single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). Suitable leucine zipper sequences include the jun and fos leucine zippers taught by Kostelney et al., J. Immunol., 148: 1547-1553 (1992) and the GCN4 leucine zipper disclosed in U.S. Pat. No. 6,025,158.

The IL-8 protein modulator may also be administered with a second therapeutic agent. The second therapeutic agent may be an antiviral agent such as ribavirin, amantidine, rimantidine, Relenza, Tamiflu, BTA 188, RWJ-270210 (BCX-1812), sICAM-1, tlCAM453, Pleconaril or AG 7088. It may also be an antihistamine, such as Benadryl, chlorpheneramine and salts thereof, brompheneramine or salts thereof, etc., a decongestant, such as phenylpropanolamine and salts thereof, pseudoephedrine or salts thereof; steroids, such as dexamethasone, prednisone, or prednisolone, etc., various antibiotics, such as the quinolones, cephalosporins, β-lactamase inhibitors, etc. or anti-inflammatory agents, such as CSAIDs, a COX-1 or COX-2 inhibitor, ASA, or indomethacin, etc.

The IL-8 protein modulator can be used in the manufacture of a medicine for the prophylactic or therapeutic treatment of symtoms or sequelae of viral infection in a human, or other mammal, which is exacerbated or caused by excessive or unregulated IL-8 cytokine production by such mammal's cell, such as, but not limited to monocytes and/or macrophages, or other chemokines which bind to the IL-8 α•or β receptor, also referred to as the type I or type II receptor.

Accordingly, the present invention provides a method of treating a viral infection, wherein the chemokine is one which binds to an IL-8 α or β receptor and which method comprises administering an effective amount of an IL-8 protein modulator. In particular, the chemokines are IL-8, GROα, GROβ, GROγ, NAP-2 or ENA-78.

In order to use an IL-8 protein modulator in therapy, it will normally be formulated into a pharmaceutical composition in accordance with standard pharmaceutical practice. This invention, therefore, also relates to a pharmaceutical composition comprising an effective, non-toxic amount of an IL-8 protein modulator and a pharmaceutically acceptable carrier or diluent.

The IL-8 protein modulators and pharmaceutical compositions incorporating such may be administered in conventional dosage forms prepared by combining a IL-8 protein modulator with standard pharmaceutical carriers according to conventional procedures. It will be appreciated that the form and character of the pharmaceutically acceptable carrier or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

An “effective amount” of the IL-8 protein modulator to be employed therapeutically will depend, for example, upon the therapeutic objectives, the route of administration, the type of polypeptide or antibody employed, and the condition of the patient. Accordingly, it will be necessary for a clinician to titer the dosage and modify the route of administration as required to obtain the optimal therapeutic effect. Typically, the clinician will administer the IL-8 protein modulator until a dosage is reached that achieves the desired effect. Such dosage is preferably below the amount that is toxic to the patient. The progress of this therapy is easily monitored by conventional assays.

The IL-8 protein modulators and pharmaceutical compositions incorporating such may conveniently be administered in any suitable manner, including parenteral, topical, oral or local (such as aerosol or transdermal) or any combinations thereof. Preferably, the mode of administration is parenteral, topical or local. Parenteral administration can include intravenous, intramuscular, subcutaneous, intranasal, intrarectal, intravaginal, intraperitoneal, intracerebral, intraocular, intraarterial, intrathecal or intralesional administration. If administered locally, the preferred mode is by aerosol.

As a general proposition, the initial pharmaceutically effective amount of an IL-8 protein modulator administered parenterally per dose will be in the range of about 0.1 to 50 mg/kg of patient body weight per day, with the typical initial range of antibody used being 0.3 to 20 mg/kg/day, more preferably 0.3 to 15 mg/kg/day. As noted above, however, these suggested amounts of antibody are subject to a great deal of therapeutic discretion.

The IL-8 protein modulators may be administered topically, that is by non-systemic administration. This includes the application of an IL-8 protein modulator externally to the epidermis or the buccal cavity, inhalation of powder or aerosol formulation, and the instillation of such a compound into the ear, eye and nose, such that the compound does not significantly enter the blood stream. In contrast, systemic administration refers to oral or parenteral administration. The daily topical dosage regimen will preferably be from 0.1 mg to 150 mg administered one to four, preferably two or three times daily.

Formulations suitable for topical administration include liquid or semi-liquid preparations suitable for penetration through the skin or airway epithelium to the site of inflammation such as liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w, but preferably will comprise less than 5% w/w, more preferably from 0.1% to 1% w/w of the formulation.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

The present IL-8 protein modulators may also be administered by locally by inhalation, that is by intranasal and oral inhalation administration, or by transdermal administration. Appropriate dosage forms for such administrations, such as an aerosol formulation, a metered dose inhaler or a transdermal patch, may be prepared by conventional techniques. The daily inhalation dosage regimen will preferably be from about 0.01 mg/kg to about 1 mg/kg per day.

The IL-8 protein modulators may also be administered in conventional dosages in combination with a known, second therapeutically active compound. The effective amount of such other agents depends on the amount of IL-8 protein modulator present in the formulation, the type of viral infection, and other factors discussed above. These are generally used in the same dosages and with administration routes as disclosed herein or from about 1 to 99% of the dosages disclosed herein.

The invention will now be described by reference to the following examples which are merely illustrative and are not to be construed as a limitation of the scope of the present invention.

### EXAMPLES

The IL-8, and GRO-α chemokine inhibitory effects of IL-8 protein modulators of the present invention are determined by the following in vitro assay:

Receptor Binding Assays:

[¹²⁵I] IL-8 (human recombinant) is obtained from Amersham Corp., Arlington Heights, Ill., with specific activity 2000 Ci/mmol. GRO-α is obtained from NEN- New England Nuclear. All other chemicals are of analytical grade. High levels of recombinant human IL-8 type α and β receptors were individually expressed in Chinese hamster ovary cells as described previously (Holmes et al., Science, 253, 1278 (1991)). The Chinese hamster ovary membranes were homogenized according to a previously described protocol (Haour et al., J. Biol. Chem., 249, 2195-2205 (1974)). Except that the homogenization buffer is changed to 10 mM Tris-HCL, 1 mM MgS04, 0.5 mM EDTA (ethylene-diaminetetra-acetic acid), 1 mM PMSF (α-toluenesulphonyl fluoride), 0.5 mg/L Leupeptin, pH 7.5. Membrane protein concentration is determined using Pierce Co. micro-assay kit using bovine serum albumin as a standard. All assays are performed in a 96-well micro plate format. Each reaction mixture contains ¹²⁵I IL-8 (0.25 nM) or ¹²⁵I GRO-α and 0.5 μg/mL of IL-8Rα or 1.0 μg/mL of IL-8Rβ membranes in 20 mM Bis-Trispropane and 0.4 mM Tris HCl buffers, pH 8.0, containing 1.2 mM MgSO4, 0.1 mM EDTA, 25 mM Na and 0.03% CHAPS. In addition, drug or compound of interest is added which has been pre-dissolved in DMSO so as to reach a final concentration of between 0.01 nM and 100 uM. The assay is initiated by addition of ¹²⁵I-IL-8. After 1 hour at room temperature the plate is harvested using a Tomtec 96-well harvester onto a glass fiber filtermat blocked with 1% polyethylenimine/0.5% BSA and washed 3 times with 25 mM NaCl, 10 mM TrisHCl, 1 mM MgSO4, 0.5 mM EDTA, 0.03% CHAPS, pH 7.4. The filter is then dried and counted on the Betaplate liquid scintillation counter. The recombinant IL-8 Rα or Type I, receptor is also referred to herein as the non-permissive receptor and the recombinant IL-8 Rβ or Type II, receptor is referred to as the permissive receptor.

Representative present compounds Examples 1 to 10⁶ have exhibited positive inhibitory activity in this assay at IC₅₀ levels<30 uM.

Chemotaxis Assay:

The in vitro inhibitory properties of these compounds are determined in the neutrophil chemotaxis assay as described in Current Protocols in Immunology, Vol. I, Suppl 1, Unit 6.12.3. Neutrophils where isolated from human blood as described in Current Protocols in Immunology Vol. I, Suppl 1 Unit 7.23.1. The chemoattractants IL-8, GRO-α, GRO-β, GRO-γ and NAP-2 are placed in the bottom chamber of a 48 multiwell chamber (Neuro Probe, Cabin John, Md.) at a concentration between 0.1 and 100 nM. The two chambers are separated by a 5 uM polycarbonate filter. When compounds of this invention are tested, they are mixed with the cells (0.001-1000 nM) just prior to the addition of the cells to the upper chamber. Incubation is allowed to proceed for between about 45 and 90 min at about 37° C. in a humidified incubator with 5% CO2. At the end of the incubation period, the polycarbonate membrane is removed and the top side washed, the membrane then stained using the Diff Quick staining protocol (Baxter Products, McGaw Park, Ill., USA). Cells which have chemotaxed to the chemokine are visually counted using a microscope. Generally, four fields are counted for each sample, these numbers are averaged to give the average number of cells which bad migrated. Each sample is tested in triplicate and each compound repeated at least four times. To certain cells (positive control cells) no compound is added, these cells represent the maximum chemotactic response of the cells. In the case where a negative control (unstimulated) is desired, no chemokine is added to the bottom chamber. The difference between the positive control and the negative control represents the chemotactic activity of the cells.

Elastase Release Assay:

The compounds of this invention are tested for their ability to prevent Elastase release from human neutrophils. Neutrophils are isolated from human blood as described in Current Protocols in Immunology Vol. I, Suppl 1 Unit 7.23.1. PMNs 0.88×10⁶ cells suspended in Ringer's Solution (NaCl 118, KCl 4.56, NaHCO3 25, KH2PO4 1.03, Glucose 11.1, HEPES 5 mM, pH 7.4) are placed in each well of a 96 well plate in a volume of 50 ul. To this plate is added the test compound (0.001-1000 nM) in a volume of 50 ul, Cytochalasin B in a volume of 50 ul (20 ug/ml) and Ringers buffer in a volume of 50 ul. These cells are allowed to warm (37° C., 5% CO2, 95% RH) for 5 min before IL-8, GRO•, GRO•, GRO• or NAP-2 at a final concentration of 0.01-1000 nM was added. The reaction is allowed to proceed for 45 min before the 96 well plate is centrifuged (800×g 5 min.) and 100 ul of the supernatant removed. This supernatant is added to a second 96 well plate followed by an artificial elastase substrate (MeOSuc-Ala-Ala-Pro-Val-AMC, Nova Biochem, La Jolla, Calif.) to a final concentration of 6 ug/ml dissolved in phosphate buffered saline. Immediately, the plate is placed in a fluorescent 96 well plate reader (Cytofluor 2350, Millipore, Bedford, Mass.) and data collected at 3 min intervals according to the method of Nakajima et al. J. Biol. Chem. 254, 4027 (1979). The amount of elastase released from the PMNs is calculated by measuring the rate of MeOSuc-Ala-Ala-Pro-Val-AMC degradation.

Rhinovirus Methods:

Cell lines and rhinovirus serotype 39 were purchased from American Type Culture Collection (ATCC). BEAS-2B cells were cultured according to instructions provided by ATCC using BEGM (bronchial epithelial growth media) purchased from Clonetics Corp. HELA cell cultures, used for detection and titration of virus, were maintained in Eagle's minimum essential media containing 10% fetal calf serum, 2 mM 1-glutamine, and 10 mM HEPES buffer (MEM).

A modification of the method reported by Subauste et al., in J. Clin. Invest. 96, 549 (1995) for in vitro infection of human bronchial epithelial cells with rhinovirus was used in these studies. BEAS-2B cells (2×105/well) were cultured in collagen-coated wells for 24 hours prior to infection with rhinovirus. Rhinovirus serotype 39 was added to cell cultures for one hour incubation at 34° C. after which inoculum was replaced with fresh media and cultures were incubated for an additional 72 hours at 34° C. Supernatants collected at 72 hours post-infection were assayed for cytokine protein concentration by ELISA using commercially available kits (R&D Systems). Virus yield was also determined from culture supernatants using a microtitration assay in HELA cell cultures (Subauste et al., supra, (1995)). In cultures treated with IL-8 inhibitors, drug was added 30 minutes prior to infection. Stocks of compounds were prepared in DMSO (10 mM drug) and stored at −20° C.

For detection of IL-8R inhibition, cultures were incubated in basal media without growth factors and additives to reduce endogenous levels of activated IL-8. Supernatants were harvested at various time points after addition of rhinovirus and concentrated. Concentrates were fractionated on Superose 6 columns. Supernatants were concentrated >50 fold using an Amicon concetrater with 5,000 mol. wt. cut off. A single injection (0.5 ml) was applied to a Superose 6 size fractionation column which was eluted at a single flow rate (0.2 ml/min). 0.5 ml fractions were collacted and assayed for Ca2+ mobilizing activity in freshly isolated human PMN loaded with FURA-2.

Results: Characterization of Chemokines Produced From RV Infected BEAS-2B Human Epithelial Cells

Under resting conditions human BEAS-2B human epithelial cells produced small quantities of at least three known human chemokines, Groα, IL-8 and ENA-78. When these cells are infected with rhinovirus production of the three ELR chemokines increases 6.6-20 fold above resting conditions (Table 1).

Table 1. Production of ELR chemokines from BEAS-2B epithelial cells under resting and infected conditions (n=6).

| Treatment | Groa (pg/ml) | IL-8 (pg/ml) | ENA-78 (pg/ml) |
|---|---|---|---|
| Control | 1568 ± 402 | 143 ± 86 | 164 ± 33 |
| HRV-39 | 12135 ± 3599 | 2870 ± 645 | 1083 ± 194 |

When HRV-39 infected epithelial cell supernatant is subject to size exclusion fractionation over a superose 6 column it results in a single peak of Ca2+ mobilizing activity when assayed using human neutrophils (Figure) or a cell line transfected with either the CXCR1 or CXCR2 receptor. The mobilization of Ca2+ in the neutrophil occurs in the same fractions as the elution of the chemokines IL-8, ENA-78 and Groa.

FIG. 1:

RV, groα, groβ, and groβ-T induced calcium mobilization in neutrophils

[image]

To determine if other chemokines are produced from HRV-39 infected BEAS-2B for which we do not have ELISA kits, BEAS-2B supernatant was assayed against other freshly isolated human peripheral cells which express a variety of chemokine receptors including CCR1, CCR2, CCR3 and CCR5. When the above active fractions derived from the BEAS-2B epithelial cells were assayed for Ca2+ mobilization using Fura-2 loaded eosinophils or peripheral blood mononuclear cells (PBLs) none of the fractions mobilized Ca2+. This indicates that chemokines besides IL-8, Groa and ENA-78 are not present at significant concentrations to promote Ca2+ mobilization.

To determine the potential for other PMN activating chemokines and for the possibility that IL-8 will activate the PMNs via CXCR1 we have determined the ability of IL-8 receptor antagonists to inhibit Ca2+ mobilization induced by groβ, concentrated BEAS-2B supernatant or active fractions derived from a superose 6 column separation in Fura-2 loaded PMNs. As can be seen from FIG. 2, compound -X dose dependently and completely inhibited RV concentrated supernatant and fractions containing the chemokines from a superose 6 column with IC50s between 1.7 and 2 nM. This IC₅₀ is similar to the IC50 derived with Gro• alone, (IC50=5 nM) and indicates that all of the Ca2+ mobilizing activity contained in the concentrate or fractions is working through the CXCR2 receptor.

[image]

FIG. 2: Inhibition of RV or Gro-beta induced calcium mobilization in human neutrophils by CXCR2 antagonist X.

To determine if the RV supernatant works solely through the CXCR2 receptor, we determined that rank correlation of a number of selective CXCR2 antagonist for there ability to inhibit Ca2+ mobilization induced by concentrated RV supernatant and Groβ. There is an excellent rank correlation for a divergent set of CXCR2 antagonist and their ability to inhibit Ca2+ mobilization induced by RV and Groβ, indicating that RV supernatant Ca2+ mobilizing activity works solely through the CXCR2 receptor on PMNs.

Inhibition of PMN chemotaxis was also determined in both the concentrated and fractionated RV sample. Chemotaxis was inhibited by specific CXCR2 antagonists as shown in FIG. 3.

[image]

These results demonstrate that BEAS-2B cells produce a variety of ELR chemokines which act through the CXCR2 receptor and which can be blocked by selective CXCR2 antagonist. Although IL-8 is present in the supernatant it also is blocked completely by the CXCR2 antagonist. This is inprobably do to its low level compared with gro•.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

1. A method of treating symptomology of the common cold as caused by human rhinovirus, other enterovirus, herpesvirus, coronavirus, influenza virus, parainfluenza virus, respiratory syncytial virus, or adenovirus infection in a human in need thereof which method comprises administering to said human an effective amount of an IL-8 protein modulator selected from the group consisting of an IL-8 antibody, an IL-8 receptor antibody, an IL-8 receptor peptide and a modified IL-8 peptide.

2. The method according to claim 1 wherein the respiratory viral infection exacerbates asthma.

3. The method according to claim 1 wherein the respiratory viral infection exacerbates chronic bronchitis.

4. The method according to claim 1 wherein the infection exacerbates chronic obstructive pulmonary disease.

5. The method according to claim 1 wherein the infection exacerbates otitis media.

6. The method according to claim 1 wherein the infection exacerbates sinusitis.

7. The method according to claim 1 wherein the respiratory viral infection is associated with a second bacterial infection, such as otitis media, sinusitis or pneumonia.

8. The method according to any one of claims 1 to 7 wherein the IL-8 protein modulator is administered with a second therapeutic agent.

9. The method according to claim 1 wherein the second therapeutic agent is selected from the group consisting of an antiviral agent, an antihistamine, a decongestant, a steroid, an antibiotic and an anti-inflammatory agent.

10. The method according to any one of claims 1-9 wherein the therapeutic agent is administered parenterally, topically or locally, or both topically and locally.

11. The method according to claim 10 wherein the compound is administered with a second therapeutic agent.

12. The method according to claim 11 wherein the second therapeutic agent is selected from the group consisting of an antiviral agent, an antihistamine, a decongestant, a steroid; an antibiotic and an anti-inflammatory agent.

13. The method according to claim 1 wherein the IL-8 protein modulator is selected from a compound disclosed in U.S. Pat. Nos. 6,025,158; 5,702,946; 5,677,426; 5,543,503; 5,440,021; PCT Publication Nos. WO99/37779; WO98/37200; WO98/24893; WO98/24884; WO97/01354; WO96/33735; WO96/34096; WO96/02576; WO95/23865; WO93/02108; WO92/04372; WO92/06697; WO95/25126; WO95/07934; WO91/08231; European Pat. No. EP 761688; or Japanese Pat. No. JP 06100595.
